(19) United States
(12) Patent Application Publication
(10) Pub. No.: US 2020/0372987 A1
(43) Pub. Date: Nov. 26, 2020

(54) SYSTEMS AND METHODS FOR AUTOMATICALLY GENERATING MEDICAL RESEARCH REPORTS

(71) Applicant: Evid Science, Inc., El Segundo, CA (US)

(72) Inventors: Matthew Michelson, La Cañada, CA (US); Michael Ross, Los Angeles, CA (US)

(21) Appl. No.: 16/421,161

(22) Filed: May 23, 2019

(51) Int. Cl. G16H 15/00; G16H 70/00; G06F 17/27

(52) U.S. Cl. CPC G16H 15/00; G06F 3/0484; G06F 17/27; G16H 70/00

(57) ABSTRACT

Systems and methods to automatically generate medical research reports are disclosed. Exemplary implementations may: obtain a set of medical results derived from a corpus of medical literature; obtain a set of directives associated with the set of medical results; present a user interface configured to facilitate automated report generation; obtain a user entry and/or selection of a subset of medical literature from the corpus of medical literature; obtain a narrative direction with an embedded directive; generate a report based on the narrative direction and execution of the embedded directive; present the report; and/or perform other operations.

FIELD OF THE DISCLOSURE

The present disclosure relates to systems and methods for automatically generating medical research reports.

### BACKGROUND

It is important for doctors and other individuals in the health field to be up-to-date on the latest, effective therapies. Some techniques are known that generate a repository of medical results based on a corpus of medical literature, which may also be refreshed as new literature is published. The medical literature may include, for example, studies, research papers, randomized controlled trials, and/or other literature. Some techniques may utilize artificial intelligence systems to extract details from the literature and represent the extracted information in a structured form providing access to the medical results by both machines and people. By automatically extracting outcomes data, sometimes called results or evidence, from the medical literature, such a structured data set of results/evidence may be created at a scale covering a multitude of the published literature.

### SUMMARY

One aspect of the present disclosure relates to a system configured to automatically generate medical research reports. A set of medical results derived from a corpus of medical literature may be obtained. Based on the set of medical results and using “mail merge” like directives (e.g., computer instructions represented by short-hand text), users may automatically create evidence-based reports (reports backed by medical literature). For example, a user interface may provide the functionality for a user to select, from the corpus, the literature they are interested in. The user may then write the “content” of the report. The content may include narrative directions for generating the report. The narrative directions may include embedded directives and natural language text provided by the user. The report may be generated by executing the embedded directives where the results of the execution of the embedded directives may be placed in the narrative directions in place of the embedded directive. The reports may be exported to various formats such as PDF, Word®, PowerPoint®, and/or other formats.

One or more implementations of a system configured to automatically generate medical research reports may include one or more hardware processors configured by machine-readable instructions. The processor(s) may be configured to obtain a set of medical results derived from a corpus of medical literature. The set of medical results may be represented by values of attributes. The processor(s) may be configured to obtain a set of directives associated with the set of medical results. An individual directive may correspond to instructions for utilizing medical results to generate reports.

The processor(s) may be configured to present a user interface configured to facilitate automated report generation. The user interface may include one or more of a literature selection portion, an input portion, a report portion, and/or other portions. The literature selection portion may be configured to obtain user entry and/or selection of medical literature from the corpus of medical literature. The input portion may be configured to obtain narrative directions for generating the reports. The narrative direction may have embedded directives. The report portion may be configured to display reports generated based on the narrative directions.

The processor(s) may be configured to obtain, via the literature selection portion, a user entry and/or selection of a subset of medical literature from the corpus of medical literature. The processor(s) may be configured to obtain, via the input portion, a narrative direction with one or more embedded directives. The processor(s) may be configured to generate a report based on one or more of the narrative direction, execution of individual embedded directives, and/or other information. By way of non-limiting illustration, a result of the execution of an embedded directive may be placed in the narrative direction in place of the embedded directive. The processor(s) may be configured to present, via the report portion, the report.

As used herein, any association (or relation, or reflection, or indication, or correspondency) involving servers, processors, client computing platforms, and/or another entity or object that interacts with any part of the system and/or plays a part in the operation of the system, may be a one-to-one association, a one-to-many association, a many-to-one association, and/or a many-to-many association or N-to-M association (note that N and M may be different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to automatically generate medical research reports, in accordance with one or more implementations.

FIG. 2 illustrates a method to automatically generate medical research reports, in accordance with one or more implementations.

FIG. 3 illustrates a user interface, in accordance with one or more implementations.

FIG. 4 illustrates a user interface, in accordance with one or more implementations.

FIG. 5 illustrates a user interface, in accordance with one or more implementations.

FIG. 6 illustrates a user interface, in accordance with one or more implementations.

### DETAILED DESCRIPTION

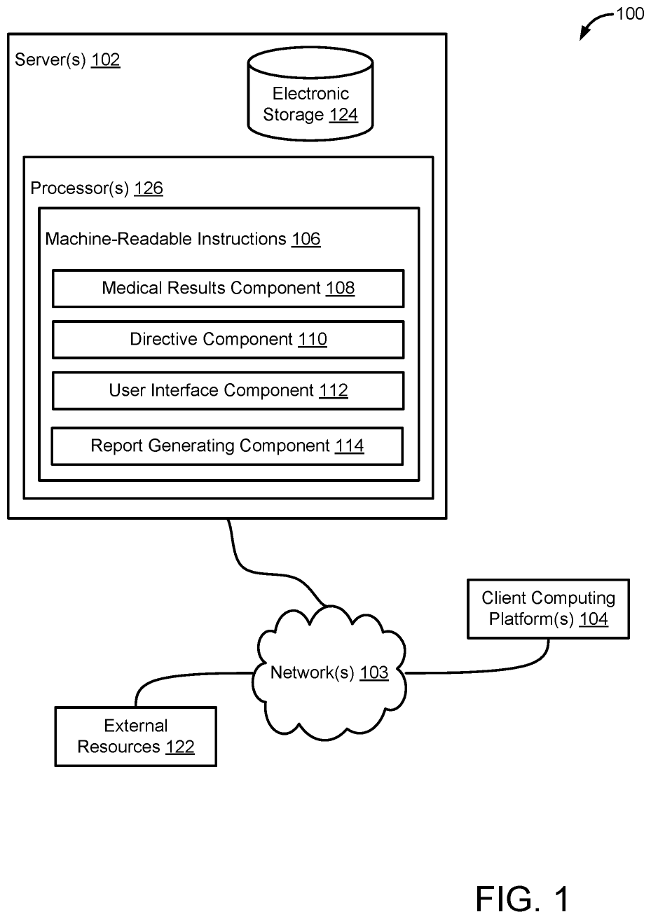

FIG. 1 illustrates a system 100 configured to automatically generate medical research reports, in accordance with one or more implementations. A set of medical results derived from a corpus of medical literature may be obtained. A user interface may allow a user to select, from the corpus, the literature they are interested in. The user may then write the “content” of a report. The content may include narrative directions for generating the report. The narrative directions may include one or more embedded directives and natural language text provided by the user. The report may be generated by executing the embedded directive(s) where the results of the execution of the embedded directive(s) may be placed in the narrative directions in place of the embedded directive. The various directives available for use may be predefined by a system administrator and provide a wide variety of functionality. In some implementations, execution of directive may result in a substitution of information present in the medical literature with the directive. In some implementations, execution of a directive may result in the generation of content, including one or more of visualizations, results of computations utilizing information in the medical literature, and/or other content. As the corpus is added to and/or updated (changed and/or modified), the generated reports may be updated to reflect the current state of the corpus of medical literature.

In some implementations, system 100 may include one or more of one or more servers 102, one or more client computing platforms 104, external resources 122, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via individual ones of the client computing platform(s) 104.

Server(s) 102 may be configured by machine-readable instructions 106. Machine-readable instructions 106 may include one or more instruction components. Executing the machine-readable instructions 106 may cause server(s) 102 to facilitate automated report generation. The instruction components may include computer program components. The instruction components may include one or more of medical results component 108, directive component 110, user interface component 112, report generating component 114, and/or other instruction components.

The medical results component 108 may be configured to obtain a set of medical results derived from a corpus of medical literature. The medical results may have been derived from a corpus of medical literature via artificial intelligence and/or other techniques. The set of medical results may be stored in a structured database. The set of medical results may be structured by virtue of the medical results being represented by values of pre-defined and/or derived attributes. The attributes may include one or more of an intervention attribute, a group attribute, an outcome attribute, a problem attribute, a context attribute, and/or other attributes.

A value of an intervention attribute may specify intervention information. Intervention information may include one or more of a drug used, an amount of a drug administered, a time period over which a drug was administered, a procedure performed, and/or other information.

A value of a group attribute may specify patient-level information and/or other information. The patient-level information may include one or more of age, age range, demographic, inclusion and/or exclusion study criteria, sex, and/or other information.

A value of an outcome attribute may specify outcome information. The outcome information may include one or more of a result of treatment, a time period in which a certain result was obtained, and/or other information. By way of non-limiting illustration, a result may include one or more of an adverse event, remission, hospitalization, and/or other information.

A value of a problem attribute may specify problem information. Problem information may include one or more of a disease present, disease study population criteria, and/or other information.

A value of a context attribute may specify information derived either directly and/or indirectly from the medical literature from which the medical results were derived. Directly derived may refer to information present on the face of the medical literature. Indirectly derived may refer to information that may be systematically and/or logically determined from the information present on the face of the medical literature. By way of non-limiting illustration, a value of a context attribute may include one or more of a citation to medical literature, a link to the literature, relative risk (e.g., a ratio of the probability of an event occurring in the exposed group versus the probability of the event occurring in the non-exposed group), a classification of the medical literature (e.g., random control study, clinical trial, etc.), a generated sentence, and/or other information. A sentence may be generated by taking discrete information (e.g., individual words, numbers, and/or other content) and generating a natural language sentence.

It is noted that the above descriptions of various attributes having values defining medical results within a database are for illustrative purposes only and not to be considered limiting. Instead, those skilled in the art may recognize other values of these attributes and/or other attributes which may be used to define the stored data.

Directive component 110 may be configured to obtain a set of directives associated with the set of medical results. The individual directives in the set of directives may be predefined by a system administrator and/or other users. The set of directives may be stored in, and/or obtained from, electronic storage 124 and/or other storage location. An individual directive may correspond to computer program instructions for utilizing medical results from the set of medical results to generate reports. The computer program instructions may include commands.

In some implementations, the directives may be defined by different classes of directives. The different classes may relate to the type of instructions which are carried out in response to execution of the directives. The different classes may include one or more of a value class, an output class, and/or other classes.

Directives of the value class may include instructions to obtain one or more of the values of the attributes. The one or more of the values of the attributes may include information present on the face of the medical literature. The one or more of the values of the attributes may include information derived from information present on the face of the medical literature.

Directives of the output class may include instructions to generate content from one or more of the values of the attributes. In some implementations, the content may include a visualization of one or more of the values of the attributes and/or other content. A visualization may include one or more of a graph, a chart, a heat map, a table, a plot, and/or other visualization. By way of non-limiting illustration, a visualization may include one or more of a comparison table, a bar chart showing how the intervention performed, an odds ratio plot visualization, a timeline of how interventions change over time, and/or other visualizations.

In some implementations, the content generated from directives of the output class may include one or more results of one or more computations which utilize one or more of the values of the attributes. For example, computations may include one or more of relative risk, log odds, the “number needed to treat” (a statistical measure), the DerSimoninan and Laird random effects model estimate of relative risk, and/or other computations.

User interface component 112 may be configured to present a user interface configured to facilitate automated report generation. The user interface may include one or more user interface portions. By way of non-limiting example, the user interface may include one or more of a literature selection portion, an input portion, a report portion, and/or other portions. In some implementations, the user interface may display one or more of the portions in a single display window, may display different portions in different display windows of the user interface, and/or have other display characteristics. The user interface may be scrollable.

The literature selection portion may be configured to obtain user entry and/or selection of a subset of the medical literature from the corpus of medical literature. In some implementations, entry and/or selection of the subset of the medical literature may include entry and/or selection of individual ones of the medical literature papers to include in the subset (e.g., by name, author, ISBN, and/or other considerations). In some implementations, entry and/or selection of the subset of the medical literature may include entry and/or selection of a search query for medical literature. The subset of medical literature may include medical literature that matches and/or is associated with the search query. The search queries may include values of attributes.

The input portion may be configured to obtain narrative directions for generating the reports. The narrative direction may include natural language text and embedded directives. The directives may be considered “embedded” by virtue of being in situ within the natural language text. In some implementations, the embedded directives may be distinguished from the natural language text by distinct alphanumeric characters. The distinct alphanumeric characters may include one or more of brackets, double brackets, and/or other distinct alphanumeric characters. The brackets may include one or more of round brackets, square brackets, box brackets, braces, curly brackets, and/or other brackets. The individual directives may be associated with short-hand descriptions of the instructions associated with individual directives. The users may input the short-hand descriptions and utilize the distinct alphanumeric characters to provide the embedded directives within the narrative directions.

By way of non-limiting illustration, a directive may be represented by “{{short-hand description}}” where “short-hand description” is one of the short-hand descriptions of the directives. The following is a non-limiting list illustrating various directives as represented by short-hand descriptions and the corresponding instructions: {{num_papers}}—the number of medical literature included in the report; {{outcome}}—one or more outcomes in the selected subset of medical literature; {{intervention}}—one or more interventions (e.g., drugs, procedures, placebos, etc.) in the selected subset of medical literature; {{problem}}—one or more problems (e.g., disease, etc.) in the selected subset of medical literature; {{disease}}—same as {{problem}}; {{compare_table}}—takes the selected subset of medical literature, and for each result, adds it as a row of a table, with columns for each attribute associated with that row (e.g, numeric result, outcome, etc.); {{expand_to_sentences}}—takes the selected subset of medical literature, and for each result or pair of results, generates a sentence describing that result using a template; {{outcome_comparison}}—creates a visualization of a bar chart showing how the intervention performed, for each paper in the selected subset of medical literature; {{relative_risk}}—computes the relative risk value, 95% confidence bounds, and p-value from the selected subset of medical literature (all statistical measures); {{log_odds}}—computes the log odds ratios and variances from the selected subset of medical literature (statistical measures); {{nnt}}—computes the number needed to treat (a statistical measure), from the selected subset of medical literature.

The report portion may be configured to display reports generated based on the narrative directions. The report portion may provide the functionality to export the reports to various formats such as PDF, Word®, PowerPoint®, and/or other formats.

By way of non-limiting illustration, user interface component 112 may be configured to obtain, via the literature selection portion, user entry and/or selection of a subset of medical literature from the corpus of medical literature. The user interface component 112 may be configured to obtain, via the input portion, a narrative direction with one or more embedded directives. The input portion may include a user interface element, such as a virtual button, to initiate a generation of a report.

Report generating component 114 may be configured to generate report based on one or more of the narrative directions, execution of the embedded directive(s), and/or other information. A result of the execution of an embedded directive (e.g., the instructions associated with the directive) may be placed in a narrative direction in place of the embedded directive. A position of an embedded directive, and/or the instructions of the directive, may provide context for the layout/presentation of the generated report. Context may refer to the how to position of the result of the execution of the directives in the generated portion. Different position may include one or more of in-line with natural language text, in a new paragraph and/or new line or sentence in the report, in a new (e.g., subsequent) page of the report, and/or other considerations. For example, a directive may include instructions for the generation of a table and/or other visualization. The directive may be positioned in a narrative direction in-line with a natural language sentence. However, when generating the report, by virtue of the directive resulting in a table (as opposed to solely text-based output), the layout of the report may position the table (or other visualization) it is own line or paragraph of the report (as opposed to in-line with the natural language sentence). User interface component 112 may be configured to present, via the report portion, the generated reports.

It is noted that the corpus of medical literature may be updated as new medical literature is produced and added to the corpus and/or existing medical literature within the corpus is updated and/or changed. The user interface component 112 may be configured to automatically update generated reports based on changes to selected literature and/or addition of new literature.

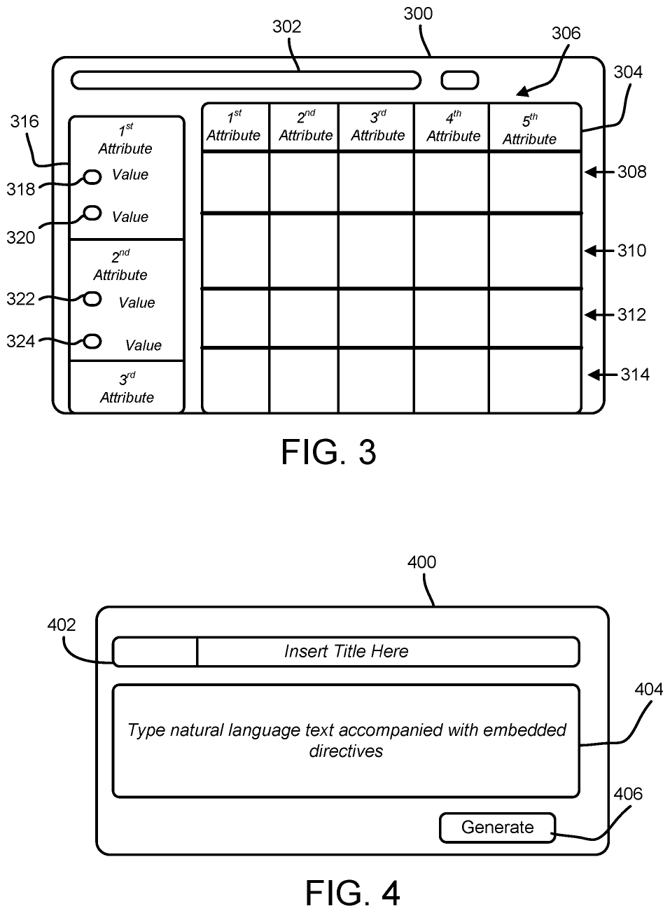

FIG. 3 illustrates an exemplary literature selection portion of a user interface 300 configured to obtain user entry and/or selection of medical literature from the corpus of medical literature, in accordance with one or more implementations. The user interface 300 may be further divided into additional user interface portions. By way of non-limiting illustration, user interface 300 may include one or more of an input portion 302, a display portion 304, a filter portion 316, and/or other portions.

The input portion 302 may comprise a text input field and/or other user interface element. The input portion 302 may be configured to obtain text-based input to search for individual papers in the medical literature and/or provide a query to search for a subset of the medical literature. The display portion 304 may comprise a display window configured in a grid comprising attribute-named columns 306 and a set of rows 308, 310, 312, 314, and/or other rows. The rows may include cells specifying individual values of corresponding attributes named in the columns 306. Individual rows may correspond to individual paper in the medical literature which matches the user queries and/or the particular paper searched for.

The filter portion 316 may include a set of user-selectable filter elements 318, 320, 322, 324, 324, and/or other elements which may be associated with a set of values (labeled generically as “value” in the figure for illustrative purposes) of individual attributes included in the attributed-named columns. The filter portion 316 may be configured to obtain user entry and/or selection of the individual user-selectable filter elements to refine the search results. Here, the set of user-selectable filter elements 318-324 are shown as individual check-boxes, however, other user interface elements may be utilized.

The user may provide entry and/or selection of a subset of medical literature shown in the display portion 304 to be included in their report generation. The selection of papers they are interested in may be facilitated by one or more selection techniques (e.g., via check-boxes, highlighting, and/or other techniques).

FIG. 4 illustrates an input portion of a user interface 400 configured to obtain narrative directions for generating the reports, in accordance with one or more implementations. The input portion of the user interface 400 may include one or more user interface elements. The user interface element may include one or more of a first text input portion 402 for specifying a title of the report to be generated and/or a category of the report to be generated, a second text input portion 404 for providing narrative directions for generating the report, a report generation initiation element 406 (e.g., a virtual button), and/or other user interface elements.

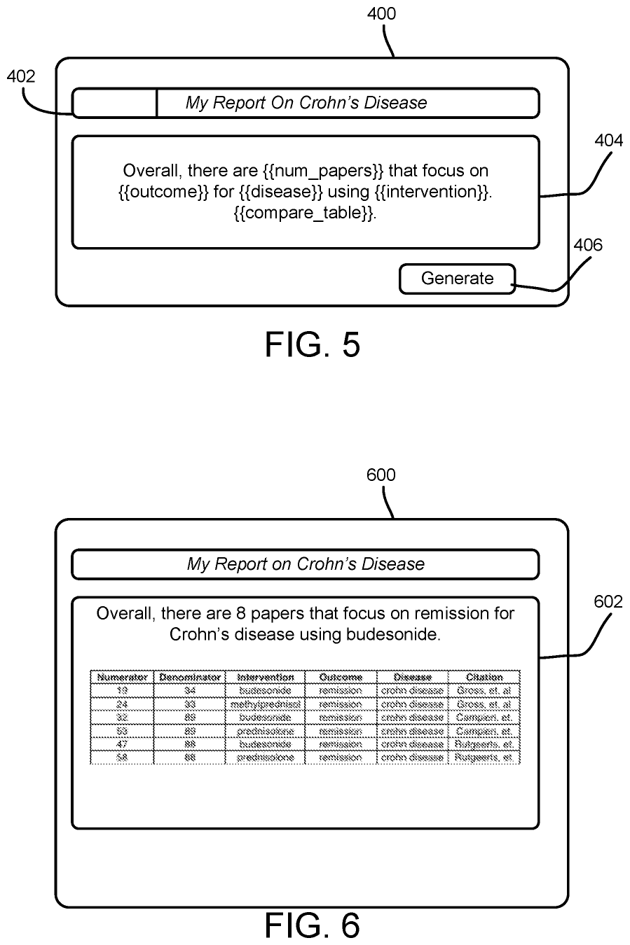

FIG. 5 illustrates the input portion of a user interface 400 showing an example of a narrative direction with embedded directives input into second text input portion 404. As shown, the narrative direction includes natural language text, e.g., “Overall, there are . . . that focus on . . . for . . . using,” and embedded directives, e.g., {{num_papers}}, {{outcome}}, {{disease}}, {{intervention}}, and {{compare_table}}.

FIG. 6 shows a report portion of a user interface 600, in accordance with one or more implementations. The report portion includes report 602 generated based on the narrative directions (see FIG. 5). In particular, the results of the execution of the directives are placed in the natural language text in place of the directives. For example, {{num_papers}} is replaced by the number of papers selected, {{outcome}} is replaced by the outcome discussed within the selected papers, {{disease}} is replaced by the disease discussed in the selected papers, {{intervention}} is placed by the intervention discussed in the selected papers, and {{compare_table}} is replaced with a comparison table generated using the information in the selected papers.

Returning to FIG. 1, in some implementations, server(s) 102, client computing platform(s) 104, and/or external resources 122 may be operatively linked via one or more electronic communication links. For example, such electronic communication links may be established, at least in part, via network(s) 103 such as the Internet and/or other networks. It will be appreciated that this is not intended to be limiting, and that the scope of this disclosure includes implementations in which server(s) 102, client computing platform(s) 104, and/or external resources 122 may be operatively linked via some other communication media.

An individual client computing platform of one or more client computing platforms 104 may include one or more processors configured to execute computer program components. The computer program components may be configured to enable an expert or user associated with the individual client computing platform to interface with system 100 and/or external resources 122, and/or provide other functionality attributed herein to client computing platform(s) 104. By way of non-limiting example, the individual client computing platform may include one or more of a desktop computer, a laptop computer, a handheld computer, a tablet computing platform, a NetBook, a Smartphone, a gaming console, and/or other computing platforms.

External resources 122 may include sources of information outside of system 100, external entities participating with system 100, and/or other resources. In some implementations, some or all of the functionality attributed herein to external resources 122 may be provided by resources included in system 100.

Server(s) 102 may include electronic storage 124, one or more processors 126, and/or other components. Server(s) 102 may include communication lines, or ports to enable the exchange of information with network(s) 103 and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 124 may comprise non-transitory storage media that electronically stores information. The electronic storage media of electronic storage 124 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 124 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 124 may include one or more virtual storage resources (e.g., cloud storage, a virtual private network, and/or other virtual storage resources). Electronic storage 124 may store software algorithms, information determined by processor(s) 126, information received from server(s) 102, information received from client computing platform(s) 104, and/or other information that enables server(s) 102 to function as described herein.

Processor(s) 126 may be configured to provide information processing capabilities in server(s) 102. As such, processor(s) 126 may include one or more of a digital processor, an analog processor, a digital circuit designed to process information, an analog circuit designed to process information, a state machine, and/or other mechanisms for electronically processing information. Although processor(s) 126 is shown in FIG. 1 as a single entity, this is for illustrative purposes only. In some implementations, processor(s) 126 may include a plurality of processing units. These processing units may be physically located within the same device, or processor(s) 126 may represent processing functionality of a plurality of devices operating in coordination. Processor(s) 126 may be configured to execute components 108, 110, 112, and/or 114, and/or other components. Processor(s) 126 may be configured to execute components 108, 110, 112, and/or 114, and/or other components by software; hardware; firmware; some combination of software, hardware, and/or firmware; and/or other mechanisms for configuring processing capabilities on processor(s) 126. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 126 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 126 may be configured to execute one or more additional components that may perform some or all of the functionality attributed below to one of components 108, 110, 112, and/or 114.

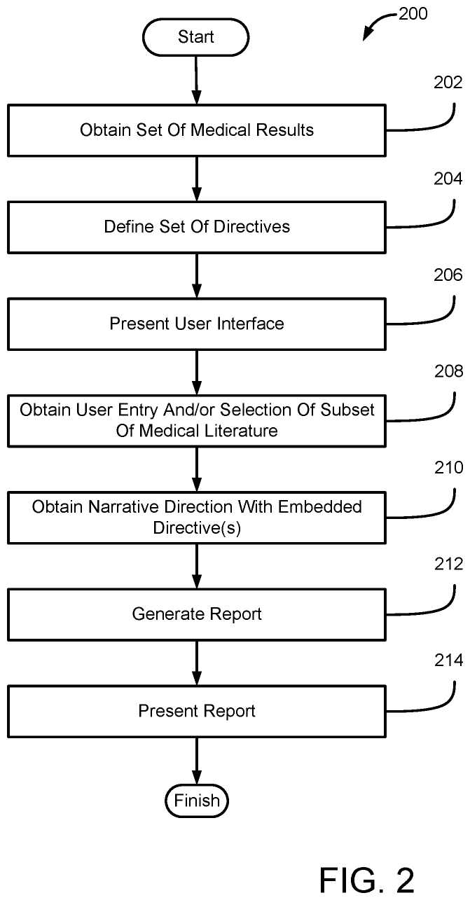

FIG. 2 illustrates a method 200 to automatically generate medical research reports, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include obtaining a set of medical results derived from a corpus of medical literature. The set of medical results may be represented by values of attributes. Operation 202 may be performed by one or more hardware processors configured by machine-readable instructions including a component that is the same as or similar to medical results component 108, in accordance with one or more implementations.

An operation 204 may include obtaining a set of directives associated with the set of medical results. An individual directive may correspond to instructions for utilizing the set of medical results to generate reports. Operation 204 may be performed by one or more hardware processors configured by machine-readable instructions including a component that is the same as or similar to directive component 110, in accordance with one or more implementations.

An operation 206 may include presenting a user interface configured to facilitate automated report generation. The user interface may include one or more of a literature selection portion, an input portion, a report portion, and/or other portions. The literature selection portion may be configured to obtain user entry and/or selection of medical literature from the corpus of medical literature. The input portion may be configured to obtain narrative directions for generating the reports. The narrative direction may have one or more embedded directives. The report portion may be configured to display reports generated based on the narrative directions. Operation 206 may be performed by one or more hardware processors configured by machine-readable instructions including a component that is the same as or similar to user interface component 112, in accordance with one or more implementations.

An operation 208 may include obtaining, via the literature selection portion, a user entry and/or selection of a subset of medical literature from the corpus of medical literature. Operation 208 may be performed by one or more hardware processors configured by machine-readable instructions including a component that is the same as or similar to user interface component 112, in accordance with one or more implementations.

An operation 210 may include obtaining, via the input portion, a narrative direction with an embedded directive and/or other embedded directives. Operation 210 may be performed by one or more hardware processors configured by machine-readable instructions including a component that is the same as or similar to user interface component 112, in accordance with one or more implementations.

An operation 212 may include generating a report based on one or more of the narrative direction, execution of one or more embedded directives, and/or other information. A result of the execution of an embedded directive may be placed in the narrative direction in place of the embedded directive. Operation 212 may be performed by one or more hardware processors configured by machine-readable instructions including a component that is the same as or similar to report generating component 114, in accordance with one or more implementations.

An operation 214 may include presenting, via the report portion, the report. Operation 214 may be performed by one or more hardware processors configured by machine-readable instructions including a component that is the same as or similar to user interface component 112, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to automatically generate medical research reports, the system comprising:
one or more physical processors configured by machine-readable instructions to:
obtain a set of medical results derived from a corpus of medical literature, the set of medical results being represented by values of attributes;
obtain a set of directives associated with the set of medical results, an individual directive corresponding to instructions for utilizing medical results from the set of medical results to generate reports;
effectuate presentation of a user interface configured to facilitate automated report generation, the user interface including a literature selection portion, an input portion, and a report portion, the literature selection portion being configured to obtain user entry and/or selection of medical literature from the corpus of medical literature, the input portion being configured to obtain narrative directions for generating the reports, the narrative direction having embedded directives, the report portion being configured to display reports generated based on the narrative directions;
obtain, via the literature selection portion, a user entry and/or selection of a subset of medical literature from the corpus of medical literature;
obtain, via the input portion, a narrative direction with an embedded directive;
generate a report based on the narrative direction and execution of the embedded directives, such that a result of the execution of the embedded directive is placed in the narrative direction in place of the embedded directive; and
effectuating, via the report portion, presentation of the report.

2. The system of claim 1, wherein the set of directives includes directives of different classes, the different classes including a value class and an output class, directives of the value class including instructions to obtain one or more of the values of the attributes, and directives of the output class including instructions to generate content from one or more of the values of the attributes.

3. The system of claim 2, wherein the one or more of the values of the attributes obtained in response to execution of the directives of the value class includes information present on the face of medical literature and/or information derived from the information present on the face of medical literature.

4. The system of claim 2, wherein the content includes a visualization of one or more of the values of the attributes.

5. The system of claim 4, wherein the visualization includes one or more of a graph, a plot, a table, or a chart.

6. The system of claim 2, wherein the content includes a result of a computation utilizing one or more of the values of the attributes.

7. The system of claim 1, wherein the narrative directions include natural language text.

8. The system of claim 7, wherein the embedded directives are embedded by virtue of being in situ within the natural language text.

9. The system of claim 8, wherein the embedded directives are distinguished from the natural language text by distinct alphanumeric characters.

10. The system of claim 1, wherein the one or more physical processors are further configured by the machine-readable instructions to:
update the reports based on changes to selected literature and/or addition of new literature to the corpus.

11. A method for automatically generating medical research reports, the method comprising:
obtaining a set of medical results derived from a corpus of medical literature, the set of medical results being represented by values of attributes;
obtaining a set of directives associated with the set of medical results, an individual directive corresponding to instructions for utilizing medical results from the set of medical results to generate reports;
presenting a user interface configured to facilitate automated report generation, the user interface including a literature selection portion, an input portion, and a report portion, the literature selection portion being configured to obtain user entry and/or selection of medical literature from the corpus of medical literature, the input portion being configured to obtain narrative directions for generating the reports, the narrative direction having embedded directives, the report portion being configured to display reports generated based on the narrative directions;
obtaining, via the literature selection portion, a user entry and/or selection of a subset of medical literature from the corpus of medical literature;
obtaining, via the input portion, a narrative direction with an embedded directive;
generating a report based on the narrative direction and execution of the embedded directives, such that a result of the execution of the embedded directive is placed in the narrative direction in place of the embedded directive; and
presenting, via the report portion, the report.

12. The method of claim 11, wherein the set of directives includes directives of different classes, the different classes including a value class and an output class, directives of the value class including instructions to obtain one or more of the values of the attributes, and directives of the output class including instructions to generate content from one or more of the values of the attributes.

13. The method of claim 12, wherein the one or more of the values of the attributes obtained in response to execution of the directives of the value class includes information present on the face of medical literature and/or information derived from the information present on the face of medical literature.

14. The method of claim 12, wherein the content includes a visualization of one or more of the values of the attributes.

15. The method of claim 14, wherein the visualization includes one or more of a graph, a plot, a table, or a chart.

16. The method of claim 12, wherein the content includes a result of a computation utilizing one or more of the values of the attributes.

17. The method of claim 11, wherein the narrative directions include natural language text.

18. The method of claim 17, wherein the embedded directives are embedded by virtue of being in situ within the natural language text.

19. The method of claim 18, wherein the embedded directives are distinguished from the natural language text by distinct alphanumeric characters.

20. The method of claim 11, further comprising updating the reports based on changes to selected literature and/or addition of new literature to the corpus.
